(19) United States
(12) Patent Application Publication
(10) Pub. No.: US 2012/0190997 A1
(43) Pub. Date: Jul. 26, 2012

(54) MAIN STREAM GAS ANALYZING DEVICE

(75) Inventor: Christopher M. VARGA, Laguna Hills, CA (US)

(73) Assignee: CAREFUSION 2200, INC., San Diego, CA (US)

(21) Appl. No.: 13/011,391

(22) Filed: Jan. 21, 2011

(51) Int. Cl. A61B 5/08

(52) U.S. Cl. 600/532; 250/343

(57) ABSTRACT

A main stream gas analyzing device for measuring a concentration of a trace gas present in a gas stream and a method of measuring the concentration of the trace gas is disclosed. The main stream gas analyzing device includes a measuring chamber for receiving a gas flow, an infrared reflective material provided on an inner surface of the measuring chamber, an infrared radiation source directed toward the infrared reflective material of the measuring chamber and obliquely angled relative to a longitudinal axis of the measuring chamber such that infrared radiation being emitted from the infrared radiation source is reflected off of the infrared reflective material at least once, and an infrared radiation detector obliquely angled relative to the longitudinal axis of the measuring chamber to receive the reflected infrared radiation.

### FIELD OF THE INVENTION

Aspects of the present invention relate to gas analyzing devices and methods for analyzing gas components of a respiratory gas.

### BACKGROUND OF THE INVENTION

It has long been desirable to identify and monitor the concentrations of components in gas streams, and in particular, respiratory gas streams. For example, in the context of respiratory diagnostics, practitioners need a way to determine diagnostic information about a patient such as lung diffusion capacity. To obtain diagnostic data, a practitioner or system may meter a low concentration of one or more gases into a larger volume of breathable gas. The patient inhales the breathable gas containing the metered gas, and then exhales the gas. Based on the concentration of metered gas that is exhaled as compared to the concentration of metered gas that was inhaled, the practitioner obtains important information about the patient's respiratory system. Thus, it is desirable to have an apparatus and method capable of measuring a trace amount of gas present in a volume of gas.

Devices that measure and monitor the concentrations of various gas components present in a gas stream can be separated into two general categories: (1) side stream gas analyzing devices and (2) main stream gas analyzing devices. Side stream gas analyzing devices divert or draw off a portion of the patient's inhaled and exhaled respiratory gases from the gas pathway in a respiratory circuit. This portion, or gas sample, is then transported to a distal site for analysis by a side stream gas analyzing device. The analyzed gas sample is either returned to the respiratory circuit, exhausted or disposed of altogether.

Main stream gas analyzing devices are configured to use a portion of the main gas pathway in a respiratory circuit as the sampling cell. Hence, there is no diversion of any of the gases going to or coming from the patient. A main stream gas analyzing device system includes a tube or passageway, also known as a measuring chamber, in direct communication with the main gas pathway in the respiratory circuit for measuring the gas components. For example the measuring chamber may be part of the inspiratory and/or expiratory path of a ventilator, or the measuring chamber may be a device a patient inhales and exhales directly into. As the patient's respiratory gases travel through the measuring chamber, the desired gas species are monitored.

Side stream gas analyzing devices have several advantages such as the ability to simultaneously analyze a plurality of gases comprising the gas sample; lack of device weight and size constraints associated with the patient circuit/interface; the ability to correct for gas pressure changes and the presence of interfering gases; and the ability to self-calibrate. Furthermore, side stream analysis allows for a more detailed analysis of the sample, including measurement of trace gas species present in the gas stream. On the other hand, diverting a portion of the patient's respiratory gases to use as the gas sample and transporting the gas sample to a distal site for the actual analysis causes a variety of issues that diminish the accuracy of the measurements. For example, signal delay due to the length of sample line from the source to the analyzers; distortion of the gas within the aforementioned gas sample line; mixing of sample gases within the aforementioned gas sample line; and difference in temperature and humidity between the sample site and the analyzing device. Furthermore, side stream analysis requires a means to move the gas sample from the sample site to the analyzer and often requires pumps, sample drying tubes, and other pneumatic components which add to the complexity and cost. The bulkiness of side stream analyzing devices makes portability difficult as well.

Main stream devices similarly have advantages and disadvantages. Advantages include: (1) no distortion of gas samples because there is no diversion from nor interference with the respiratory circuit; (2) continuous monitoring; (3) fast response; (4) reduced complexity and reduction of pneumatic components associated with side stream measurements, and (5) negligible time delay from sampling to measurement display. On the other hand, in known main stream devices, the physical constraints of having to measure directly within the main gas pathway of the system or device limit the opportunity to make detailed measurements, particularly for gases of interest that are present in very low concentrations.

U.S. Pat. No. 6,534,769 discloses a main stream gas analyzing device having an airway adapter and an external infrared radiation source. The infrared source radiation is contacted with reflective surfaces external to the airway adapter, which direct the infrared radiation into and out of the airway adapter via opposing windows. Thus, the internal path of travel of the infrared radiation is equal to the width of the airway adapter. After the infrared radiation exits the airway adapter, it is again reflected from a second external reflective surface towards an infrared radiation detector. While the disclosed apparatus may be suitable for use in measuring gases that are present in moderate to high concentrations or which have strong infrared absorption profiles such as carbon dioxide, it is not suitable for detecting gases at very low concentrations (trace gases) because the path of travel of the internal infrared radiation is relatively short, i.e., is only as long as the width of the airway adapter. Thus, the infrared radiation device disclosed, while providing trustworthy measurements for some gases will not provide trustworthy measurements for gases with weaker infrared absorption profiles or low concentrations due to the short infrared radiation travel path length and associated low number of encounters between the infrared radiation and the trace gas molecules.

U.S. Pat. No. 7,132,658 discloses a similar main stream gas analyzing device where infrared radiation is directed into a gas flow and then externally reflected toward an infrared radiation detector. However, as with the previously-discussed patent, the internal infrared radiation travel path is equal to the width of the gas passageway, and is similarly too short to adequately measure trace gases in order to provide reliable results.

U.S. Pat. No. 7,235,054 discloses a similar main stream gas analyzing device where infrared radiation is directed through a gas flow and then received by an infrared detector. Again, the internal path of travel of the infrared radiation is equal to the width of the gas passageway, and is therefore too short to adequately and reliably measure trace gases.

Thus, there is a need in the art for a main stream gas analyzing device system that is capable of reliably measuring gases present in low concentrations in a gas stream, alternatively referred to herein as trace gases.

### SUMMARY OF THE INVENTION

The present invention provides a main stream gas analyzing device, comprising a measuring chamber for receiving a gas flow, the measuring chamber being coupled with a main stream path of a respiratory device, an infrared reflective material provided on an inner surface of the measuring chamber, an infrared radiation source directed toward the infrared reflective material of the measuring chamber and obliquely angled relative to a longitudinal axis of the measuring chamber such that infrared radiation being emitted from the infrared radiation source is reflected off of the infrared reflective material at least once, and an infrared radiation detector obliquely angled relative to the longitudinal axis of the measuring chamber to receive the reflected infrared radiation.

The present invention also provides a method of determining a concentration of at least one trace gas present in a gas stream, the method comprising coupling a measuring chamber for receiving the gas stream to a main stream path of a respiratory device, the measuring chamber having an infrared reflective material provided on an inner surface of the measuring chamber, passing the gas stream through the measuring chamber, emitting infrared radiation through the gas stream and towards the infrared reflective material at an oblique emitting angle relative to a longitudinal axis of the measuring chamber such that the infrared radiation is reflected off of the infrared reflective material at least once, measuring the reflected infrared radiation via an infrared radiation detector obliquely angled relative to the longitudinal axis of the measuring chamber to receive the reflected infrared radiation, and determining the amount of the at least one trace gas present in the gas stream based on the infrared radiation measurement.

The above and still other advantages of the invention will be apparent from the detailed description and drawings. What follows are one or more preferred embodiments of the present invention.

### DESCRIPTION OF THE DRAWINGS

FIG. 1a is a first exemplary aspect of a main stream gas analyzing device of the present invention;

FIG. 1b is a second exemplary aspect of a main stream gas analyzing device of the present invention;

FIG. 2a is a third exemplary aspect of a main stream gas analyzing device of the present invention; and

FIG. 2b is a fourth exemplary aspect of a main stream gas analyzing device of the present invention.

### DETAILED DESCRIPTION OF THE INVENTION

The present invention provides a main stream gas analyzing device and a method of determining a concentration of at least one trace gas present in a gas stream. It is to be understood that the term gas is intended to include any gas suitable for use with the following disclosure. For example, the gas stream may comprise oxygen, air, or any other breathable gas. The term trace gas is intended to include any gas in the gas stream that makes up about 1% or less by volume of the gas stream. As used herein, the term main stream is intended to mean a portion of the main respiratory circuit or device gas passageway not involving the diversion of any of the gases going to or coming from the patient. The gas analyzing device includes a measuring chamber having an infrared reflective material provided on an inner surface, an infrared radiation source directed toward the infrared reflective material, and an infrared radiation detector angled to receive the reflected infrared radiation. The method of determining a concentration of at least one trace gas present in a gas stream includes passing a gas stream having at least one trace gas through a measuring chamber having an infrared reflective surface, emitting infrared radiation through the gas stream towards the infrared reflective surface such that he infrared radiation is reflected, measuring the intensity of the reflected infrared radiation via an infrared radiation detector, and determining an amount of the at least one trace gas present in the gas stream. Thus, the present invention effectively and easily allows a practitioner to determine an amount of trace gas present in a respiratory gas, while avoiding the disadvantages of a side stream analyzing device.

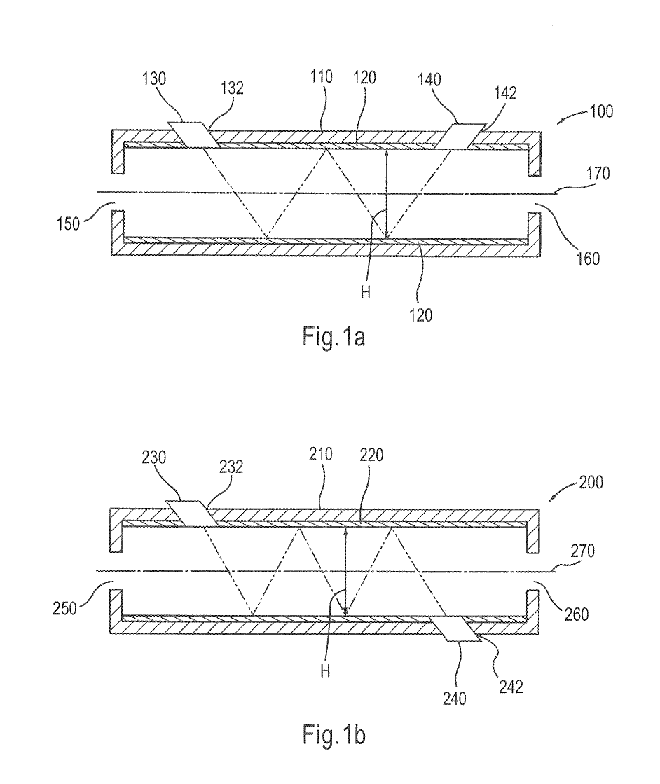

FIGS. 1-4 illustrate several exemplary aspects of the main stream gas analyzing device of the present invention. The main stream gas analyzing device includes a measuring chamber where a gas sample will be subject to examination. The measuring chamber includes an inlet and an outlet. The inlet is in communication with a respiratory gas stream. In an aspect of the present invention, the gas stream may be a respiratory gas. More particularly, the gas stream may be a gas being inhaled or exhaled by a patient which contains at least one trace gas. In one aspect the inlet may be connected with a respiratory circuit, while in another aspect, the inlet may be in direct communication with the patient's mouth. In either aspect, the gas analyzing device is part of the main stream line because the gas is not drawn off through secondary pathways or removed and carried to an external location for analysis. The outlet of the measuring chamber may be in communication with a respiratory circuit, such as when the patient is on a ventilator. In another aspect, such as when the inlet is in direct communication with the patient's mouth, the outlet may be in communication with the atmosphere. In another aspect, the outlet may be in communication with a gas delivery device. Essentially, the gas stream containing a trace gas enters through the inlet, passes through the measuring chamber where a measurement is taken, and then exits through the outlet. In yet another aspect, the gas stream containing a trace gas enters through the outlet, passes through the measuring chamber where a measurement is taken, and then exits through the inlet.

The measuring chamber further includes a reflective material provided on an inner surface capable of reflecting infrared radiation, an infrared radiation source for providing infrared radiation, and an infrared radiation detector for receiving infrared radiation. The reflective surface may comprise any material having reasonably high reflectivity to infrared radiation, preferably around 90% or greater, in the target wavelength range for the gases being measured. For example, the reflective material may be gold, silver, aluminum, aluminum silicon oxide, or aluminum magnesium fluoride. The infrared reflective material may be applied to an inner surface of the measuring chamber using any suitable means, such as coating the material onto the inner surface, securing a plate of material onto the inner surface, or forming the measuring chamber itself from the reflective material.

The infrared radiation source is directed so that the emission of infrared radiation opposes the reflective surface. The infrared radiation source is also angled with respect to a longitudinal axis of the chamber so that the infrared radiation travels obliquely to the longitudinal axis. Because of the angle of emission, the infrared radiation will bounce off of the reflective surface at an angle that is also oblique with respect to the longitudinal axis. After reflecting off of the reflective surface the infrared radiation travels towards the opposing surface. Depending on the angle of reflection, the infrared radiation may be further reflected off the opposing surface. Each reflection of the infrared radiation increases the internal travel path of the infrared radiation, thereby increasing the encounter rate of infrared radiation with trace gas molecules.

After the infrared radiation has reflected off of the reflective surface at least one time, the infrared radiation is received by an infrared radiation detector. Like the infrared radiation source, the infrared radiation detector is angled obliquely with respect to the longitudinal axis such that it is aligned with the travel path of the infrared radiation. The infrared radiation detector receives the infrared radiation after it has passed through the gas stream. The present invention uses known non-dispersive infrared radiation spectroscopy techniques to determine what concentration of a particular gas species is present in the gas stream. Summarily stated, the technique involves taking advantage of the fact that gases absorb infrared energy at a peak absorbance wavelength specific to an individual gas. Thus, by emitting infrared radiation at a wavelength range including a particular peak absorbance wavelength at a known intensity, and detecting the diminished intensity by the infrared detector configured to detect the infrared radiation at the same peak absorbance wavelength after the infrared radiation has passed through the gas stream, a practitioner can correlate the intensity measurement with how much of that particular gas is present in the gas stream at the time of analysis. The technique may also incorporate a reference receiver in the infrared detector that measures infrared radiation intensity having wavelengths other than the peak absorbance wavelength associated with the particular gas being measured. By measuring intensity at wavelengths other than the peak absorbance wavelength the reference receiver acts as a dynamic zero, thereby minimizing interference in the measurement. However, it is within the scope of the invention that the measurement may be taken without having a reference receiver. Additional details of the non-dispersive infrared radiation spectroscopy technique are further described in U.S. Pat. No. 6,534,769, U.S. Pat. No. 7,132,658, and U.S. Pat. No. 7,235,054, all of which are incorporated by reference herein.

Multiple trace gases can be detected by duplicating the emitters and detectors described above. For example, the infrared radiation source can include multiple emitters, each of which emit infrared radiation at a different peak emission wavelength corresponding to a particular trace gas. Furthermore, the infrared detector can include multiple detectors, each of which is configured to measure only infrared radiation intensity having a particular peak wavelength. Thus, each detector may be configured to provide information regarding a single trace gas present in the gas stream. While the gas stream analyzing device is suitable for detecting any gas or trace gas, trace gases such as carbon monoxide, acetylene and methane provide particularly useful respiratory and cardiac diagnostic information. Trace gases such as these typically make up less than 1% and usually less than 0.5% by volume of the total gas present in the gas stream. By providing a gas stream analyzing device that increases the infrared travel path within the measurement chamber, the infrared radiation sufficiently contacts the trace gases, thereby allowing a practitioner to measure the amount of trace gases present in the gas stream. Prior referenced devices are not capable of achieving this benefit because the path of travel of infrared radiation through the gas stream is not long enough to encounter sufficient trace gas molecules to provide reliable measurements.

The method of determining a concentration of at least one trace gas present in a gas stream of the present invention uses the above-described main stream gas analyzing device. The main stream gas analyzing device is coupled to the main gas pathway of a respiratory device. This may include coupling the main stream gas analyzing device to a part of a respiratory circuit of a ventilated patient, the respiratory circuit configured to deliver gases, or may include having a patient breathe directly into the main stream gas analyzing device. A gas stream having at least one trace gas is passed through the measuring chamber of the main stream gas analyzing device. When used for diagnostic purposes, a practitioner or system may intentionally inject a small known concentration of one or more trace gases into the gas stream before the patient inhales the gas stream. The patient then inhales the gas stream having the trace gas species. The exhaled gas stream is subsequently passed through the main stream gas analyzing device.

As the gas stream passes through the measuring chamber, the infrared radiation source emits infrared radiation having a wavelength range corresponding to a peak absorbance wavelength of at least one of the trace gases. For example, the wavelength range may be 1 to 5 micrometers, although other suitable wavelengths specific to the trace gas being measured are well-known to a practitioner in the art. Because the radiation source is angled as described above, and because the inner surface of the measuring chamber reflects infrared radiation, the infrared radiation passes across a height or width of the measuring chamber at least twice before reaching the infrared detector. As the infrared radiation passes through the gas stream it comes into contact with the trace gas molecules. The infrared radiation is received by the detector, which measures the intensity of the infrared radiation. As described above, the known non-dispersive infrared radiation spectroscopy technique allows a practitioner to determine the amount of gas present in the gas stream based on the detected infrared radiation intensity.

Exemplary aspects of the present invention will now be disclosed. Referring to FIG. 1a, an exemplary main stream gas analyzing device 100 is shown. The main stream gas analyzing device 100 comprises a measuring chamber 110, an infrared radiation reflective material 120 provided on an inner surface of the measuring chamber 110, an infrared radiation source 130, and an infrared radiation detector 140. The measuring chamber 110 further includes an inlet 150 and an outlet 160 for allowing a gas stream having at least one trace gas to pass in and out of the measuring chamber. The infrared radiation source 130 is angled obliquely with respect to a longitudinal axis 170 of the measuring chamber. The oblique angle allows the infrared radiation to reflect off of the infrared radiation reflective material 120 toward an opposing side of the measuring chamber, while simultaneously traveling in a direction along the longitudinal axis 170 and towards the infrared detector 140. The degree of the emission angle may be chosen based on the number of reflections that are desired. The closer the emission angle is to perpendicular to the longitudinal axis, the more reflections will occur before the infrared radiation reaches the infrared detector. Thus, even if the length of the measuring chamber is relatively small, an angle of emission may be chosen such that the internal travel path of the infrared radiation may be several times greater than a height H of the measuring chamber. For example, the angle may be chosen so that the internal infrared travel path is at least twice or at least three times as long as the height H of the measuring chamber. It is within the scope of the invention, however, than any angle may be chosen to result in a desired infrared radiation travel path length.

In the aspect of the invention shown in FIG. 1a, the measuring chamber 110 further comprises a first aperture 132 defined in a first portion of a side wall of the measuring chamber, where the infrared radiation source 130 is disposed. In this aspect, the infrared radiation source is integral with the measuring chamber. Similarly, the measuring chamber of FIG. 1a has a second aperture 142 defined in a second portion of the side wall of the measuring chamber, where the infrared radiation detector 140 is disposed. When the infrared emitter and detector are provided in this manner, the infrared radiation does not have to pass through any windows and is entirely contained within the measuring chamber throughout the travel path. An advantage of this arrangement is that there is less chance for interference from additional components and more accurate data may be collected.

As shown in FIG. 1a, the first aperture 132 may be coplanar with the second aperture 142 in a horizontal plane. By having the apertures 132, 142 be coplanar in a horizontal plane, the apertures are essentially defined through the same side of the measuring chamber 110. Thus, because the infrared radiation source 130 and the infrared radiation detector 140 are disposed within the apertures 132, 142, the infrared radiation source 130 and the infrared radiation detector 140 may also be on the same side of the measuring chamber 110.

The method of determining a concentration of at least one trace gas present in a gas stream using the main stream gas analyzing device 100 will now be described. The angle of emission from infrared radiation source 130 may be chosen to provide a desired travel path length to the infrared radiation within the measuring chamber 110. This can be achieved by angling the infrared radiation source 130 and the infrared radiation detector 140 with respect to the longitudinal axis 170 of the measuring chamber 110. The main stream gas analyzing device 100 is coupled to a main stream gas pathway of a respiratory device or directly to a patient. A gas stream having at least one trace gas is passed through the measuring chamber 110 of the main stream gas analyzing device 100.

As the gas stream passes through the measuring chamber 110, the infrared radiation source 130 emits infrared radiation having a wavelength range corresponding to a peak absorbance wavelength of at least one of the trace gases. For example, the wavelength range may be 1 to 5 micrometers. Because the radiation source is angled as described above, and because the inner surface of the measuring chamber reflects infrared radiation, the infrared radiation passes across the height of the measuring chamber at least twice before reaching the infrared detector. As the infrared radiation passes through the gas stream it comes into contact with the trace gas molecules. The infrared radiation is received by the infrared radiation detector 140, which measures the intensity of the infrared radiation. The measuring chamber 110 may also be heated in order to avoid condensation of water vapor on the inner surfaces of the measuring chamber which can interfere with measurement of the target trace gas molecules. Controlled heating of the measuring chamber may also improve measurement stability.

Referring to FIG. 1b, an exemplary main stream gas analyzing device 200 is shown. The main stream gas analyzing device 200 is similar to the gas analyzing device 100 shown in FIG. 1a, except for the placement of the infrared radiation source 230 and the infrared radiation detector 240. The main stream gas analyzing device 200 generally comprises a measuring chamber 210, an infrared radiation reflective material 220 provided on an inner surface of the measuring chamber 210, an infrared radiation source 230, and an infrared radiation detector 240. The measuring chamber 210 further includes an inlet 250 and an outlet 260 for allowing a gas stream having at least one trace gas to pass in and out of the measuring chamber. The infrared radiation source 230 is angled obliquely with respect to a longitudinal axis 270 of the measuring chamber. The oblique angle allows the infrared radiation to reflect off of the infrared radiation reflective material 220 toward an opposing side of the measuring chamber, while simultaneously traveling in a direction along the longitudinal axis 270 and towards the infrared detector 240. As described above with respect to main stream gas analyzing device 100, the degree of the emission angle directly correlates to the length of the infrared radiation travel path.

The main stream gas analyzing device 200 differs from the main stream gas analyzing device 100 in that a first aperture 232 and a second aperture 242 defined in the sidewalls of the measuring chamber are disposed in parallel horizontal planes. By having the apertures 232, 242 disposed in parallel horizontal planes, the apertures are essentially defined through opposite side walls of the measuring chamber 210. As with the main stream gas analyzing device 100 of FIG. 1a, the infrared radiation source 230 and the infrared radiation detector 240 are disposed within the first and second apertures 232, 242, respectively. Thus, because the infrared radiation source 230 and the infrared radiation detector 240 are disposed within the apertures 232, 242, the infrared radiation source 230 and the infrared radiation detector 240 may also be on disposed on opposite side walls of the measuring chamber 210.

The method of determining a concentration a trace gas present in the gas stream is accomplished by performing essentially the same steps described above with respect to the main stream gas analyzing device 100. However, instead of the infrared radiation detector 240 receiving the infrared radiation in a common horizontal plane with the infrared radiation source 230, the infrared radiation detector 240 receives the infrared radiation in a parallel horizontal plane.

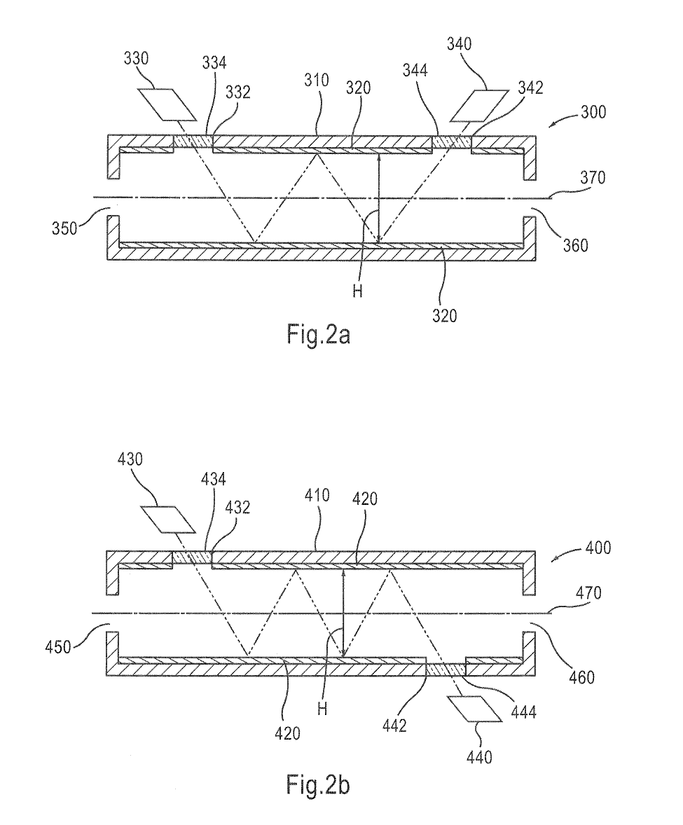

Referring to FIG. 2a, an exemplary main stream gas analyzing device 300 is shown. The main stream gas analyzing device 300 is similar to the gas analyzing device 100 shown in FIG. 1a, except for the placement of the infrared radiation source 330 and the infrared radiation detector 340. The main stream gas analyzing device 300 generally comprises a measuring chamber 310, an infrared radiation reflective material 320 provided on an inner surface of the measuring chamber 310, an infrared radiation source 330, and an infrared radiation detector 340. The measuring chamber 310 further includes an inlet 350 and an outlet 360 for allowing a gas stream having at least one trace gas to pass in and out of the measuring chamber. The infrared radiation source 330 is angled obliquely with respect to a longitudinal axis 370 of the measuring chamber. The oblique angle allows the infrared radiation to reflect off of the infrared radiation reflective material 320 toward an opposing side of the measuring chamber, while simultaneously traveling in a direction along the longitudinal axis 370 and towards the infrared detector 340. As described above with respect to main stream gas analyzing device 100, the degree of the emission angle directly correlates to the length of the infrared radiation travel path.

Furthermore, like the main stream gas analyzing device 100, the measuring chamber 310 of the main stream gas analyzing device 300 further comprises a first aperture 332 defined in a side wall of the measuring chamber and a second aperture 342 defined in a side wall of the measuring chamber. The first aperture 332 may be coplanar with the second aperture 342 in a horizontal plane. By having the apertures 332, 342 coplanar in a horizontal plane, the apertures are essentially defined through the same side of the measuring chamber 310.

The main stream gas analyzing device 300 differs from the main stream gas analyzing device 100 in that a first window 334 is disposed in the first aperture 332 and a second window 344 is disposed in the second aperture 342. Accordingly, unlike the main stream gas analyzing device 100, the infrared radiation source 330 and the infrared radiation detector 340 are not disposed in the first and second apertures 332, 342. Rather, as shown in FIG. 2a, the infrared radiation source 330 and the infrared radiation detector 340 are external to the measuring chamber 310. The windows may be made of any suitable material that is capable of allowing infrared radiation to be transmitted with reasonable efficiency. For example, the windows may be constructed of sapphire, calcium fluoride, magnesium fluoride, zinc selenide, germanium, or a synthetic material such as AMTIR-1 (amorphous material transmitting infrared radiation). The window constructions may also comprise optics and/or optical coatings which provide improved signal strength and quality. For example, the window constructions may include focusing optics, collimating optics, or anti-reflective coatings. The infrared radiation source and infrared radiation detector may be externally mounted using any known technique that would securely maintain the position and angle of each. For example, the infrared radiation source and detector may be clamped in position using a known clamping device.

Because the infrared radiation source and the infrared radiation detector is external to the measuring chamber, the infrared radiation must first pass into the measuring chamber through the first window 334 before contacting the gas molecules present in the measuring chamber. Likewise, before the reflected infrared radiation can be detected by the external infrared radiation detector, the infrared radiation must pass out of the measuring chamber through the second window 344. Thus, part of the infrared radiation path is external to the measuring chamber. The advantage of using an external infrared source and an external detector is that the measuring chamber can be easily decoupled from the respiratory device and disposed after a single use, without requiring disposal of the infrared radiation source and detector. Furthermore, the infrared source and detector may be replaced without replacing the measuring chamber.

The method of determining a concentration of a trace gas present in the gas stream using the main stream gas analyzing device 300 is accomplished by performing essentially the same steps described above with respect to the main stream gas analyzing device 100. When using the main stream gas analyzing device 300 the infrared radiation emitted from the infrared radiation source 230 is passed through the first window 332 to enter the measuring chamber 310 and the reflected infrared radiation is passed through the second window 342 to exit the measuring chamber 310. Furthermore, the method may include mounting or positioning the infrared radiation source 330 and detector 340 external to the measuring chamber.

Referring to FIG. 2b, an exemplary main stream gas analyzing device 400 is shown. The main stream gas analyzing device 400 is similar to the gas analyzing device 300 shown in FIG. 2a, except for the placement of the windows, the infrared radiation source, and the infrared radiation detector. The main stream gas analyzing device 400 generally comprises a measuring chamber 410, an infrared radiation reflective material 420 provided on an inner surface of the measuring chamber 410, an infrared radiation source 430, and an infrared radiation detector 440. The measuring chamber 410 further includes an inlet 450 and an outlet 460 for allowing a gas stream having at least one trace gas to pass in and out of the measuring chamber. The infrared radiation source 430 is angled obliquely with respect to a longitudinal axis 470 of the measuring chamber. The oblique angle allows the infrared radiation to reflect off of the infrared radiation reflective material 420 toward an opposing side of the measuring chamber, while simultaneously traveling in a direction along the longitudinal axis 470 and towards the infrared detector 440.

The main stream gas analyzing device 400 differs from the main stream gas analyzing device 300 in that a first aperture 432 and a second aperture 442 defined in the sidewalls of the measuring chamber are disposed in parallel horizontal planes. By having the apertures 432, 442 disposed in parallel horizontal planes, the apertures are essentially defined through opposite side walls of the measuring chamber 410. As with the main stream gas analyzing device 300 of FIG. 2a, a first window 434 and a second window 444 are disposed in the first and second apertures 432, 442, respectively. Accordingly, similar to the main gas stream analyzing device 300, the infrared radiation source 430 and the infrared radiation detector 440 are not disposed in the first and second apertures 432, 442. Rather, as shown in FIG. 2b, the infrared radiation source 430 and the infrared radiation detector 440 are external to the measuring chamber 410. Thus, the main stream analyzing device 400 includes the opposing apertures of the aspect shown in FIG. 1b in combination with the external placement of the infrared radiation source and detector of the aspect shown in FIG. 2a.

The method of determining a concentration of a trace gas present in the gas stream using the main stream gas analyzing device 400 is accomplished by performing essentially the same steps described above with respect to the main stream gas analyzing device 300, except that the infrared radiation is emitted and detected on opposing external sides of the measuring chamber 400.

The invention has been described herein with reference to various specific and preferred materials, embodiments and techniques. It should be understood that many modifications and variations to such materials, embodiments and techniques will be apparent to those skilled in the art within the spirit and scope of the invention. In particular, the aspects shown in FIGS. 1a-2b, for example, show a similar emission angle, similar distance between first and second apertures, and similar relative length of the measuring chamber. However, it is within the scope of the invention that the elements of the main stream gas analyzing devices may be modified and optimized depending on the gas to be measured and the desired application.

For example, it is within the scope of the invention that the measuring chamber can be shortened while altering the infrared radiation emission angle to maintain a desired infrared radiation travel path. Furthermore, the apertures, windows, infrared radiation emitter and detector, may be positioned closer or farther away from each other to optimize the infrared radiation travel path. Additionally, while FIGS. 1a-2b illustrate the measuring chamber as generally being rectangular, it is within the scope of the invention that alternative geometries may be implemented. For example, the measuring chamber may be circular or shaped in such a way that the reflections provide radiation focusing. Similarly, while the infrared radiation source and detector or windows are illustrated and described as being disposed in common or parallel horizontal planes, it is within the scope of the invention that the infrared radiation source and detector or windows are disposed in non-parallel planes. It should also be understood that the locations of the infrared radiation source and the infrared radiation detector in the main stream gas analyzing devices 100, 200, 300, and 400 can be reversed with respect to the illustrated inlet and outlet of the measuring chamber such that the infrared radiation source is closest to the outlet and the infrared radiation detector is closest to the inlet. That is, the infrared radiation may be emitted such that the infrared radiation travels in an upstream direction instead of a downstream direction. Therefore, the invention should not be limited by the above description, and to ascertain the full scope of the invention, the following claims should be referenced.

All references cited herein are hereby incorporated by reference in their entirety.

1. A main stream gas analyzing device, comprising:
a measuring chamber for receiving a gas flow, the measuring chamber being coupled with a main stream path of a respiratory device;
an infrared reflective material provided on an inner surface of the measuring chamber;
an infrared radiation source directed toward the infrared reflective material of the measuring chamber and obliquely angled relative to a longitudinal axis of the measuring chamber such that infrared radiation being emitted from the infrared radiation source is reflected off of the infrared reflective material at least once; and
an infrared radiation detector obliquely angled relative to the longitudinal axis of the measuring chamber to receive the reflected infrared radiation.

2. The main stream gas analyzing device of claim 1, wherein the infrared radiation source is disposed within a first aperture defined through a first portion of the measuring chamber.

3. The main stream gas analyzing device of claim 2, wherein the infrared radiation detector is disposed within a second aperture defined through a second portion of the measuring chamber.

4. The main stream gas analyzing device of claim 3, wherein the second aperture is coplanar with the first aperture in a horizontal plane.

5. The main stream gas analyzing device of claim 3, wherein the first aperture and the second aperture are disposed in parallel horizontal planes.

6. The main stream gas analyzing device of claim 1, wherein the measuring chamber further comprises a first window, and wherein the infrared radiation source is obliquely angled relative to the longitudinal axis of the measuring chamber such that the infrared radiation passes through the first window.

7. The main stream gas analyzing device of claim 6, wherein the measuring chamber further comprises a second window, wherein the infrared radiation source is obliquely angled relative to the longitudinal axis of the measuring chamber such that the infrared radiation passes through the second window, and wherein the infrared radiation detector is obliquely angled relative to the longitudinal axis of the measuring chamber to detect infrared radiation passing through the second window.

8. The main stream gas analyzing device of claim 7, wherein the first window is coplanar with the second window in a horizontal plane.

9. The main stream gas analyzing device of claim 7, wherein the first window and the second window are disposed in parallel horizontal planes.

10. The main stream gas analyzing device of claim 1, wherein the infrared radiation source comprises at least one infrared radiation emitter, wherein each infrared radiation emitter is configured to emit infrared radiation having a predetermined wavelength range, wherein the predetermined wavelength range contains at least one peak absorbance wavelength of a trace gas species.

11. The main stream gas analyzing device of claim 10, wherein the infrared radiation detector comprises at least one infrared radiation measuring receiver, wherein each infrared radiation measuring receiver is configured to only measure infrared radiation corresponding to one of the at least one peak absorbance wavelengths of a trace gas species.

12. The main stream gas analyzing device of claim 11, wherein the infrared radiation detector further comprises at least one reference receiver configured to measure infrared radiation at infrared wavelengths which do not include the at least one peak absorbance wavelengths of the trace gas species.

13. The main stream gas analyzing device of claim 1, wherein gas flow comprises at least one trace gas species, wherein the amount of the at least one trace gas species present in the gas flow is less than 1% by volume of the total volume of gases present in the gas flow.

14. The main stream gas analyzing device of claim 13, wherein the trace gas species is selected from the group consisting of carbon monoxide, acetylene, and methane.

15. The main stream analyzing device of claim 1, wherein the infrared radiation source is obliquely angled relative to the longitudinal axis of the measuring chamber such that infrared radiation being emitted from the infrared radiation source is reflected off of the infrared reflective material at least three times.

16. The main stream analyzing device of claim 10, wherein the predetermined wavelength range is from 1 to 5 micrometers.

17. The main stream analyzing device of claim 1, wherein the measuring chamber is heated.

18. The main stream analyzing device of claim 1, wherein the infrared reflective material comprises a material with greater than 90% reflectivity in the infrared spectrum.

19. The main stream analyzing device of claim 18, wherein the infrared reflective material is selected from the group consisting of gold, silver, aluminum, aluminum silicon oxide, and aluminum magnesium fluoride.

20. The main stream analyzing device of claim 1, wherein a distance of a path of travel of the emitted infrared radiation within the measuring chamber is greater than twice a height of the measuring chamber.

21. The main stream analyzing device of claim 1, wherein the respiratory device comprises a ventilator.

22. A method of determining a concentration of at least one trace gas species present in a gas stream, the method comprising:
coupling a measuring chamber for receiving the gas stream to a main stream path of a respiratory device, the measuring chamber having an infrared reflective material provided on an inner surface of the measuring chamber;
passing the gas stream through the measuring chamber;
emitting infrared radiation through the gas stream and towards the infrared reflective material at an oblique emitting angle relative to a longitudinal axis of the measuring chamber such that the infrared radiation is reflected off of the infrared reflective material at least once;
measuring the reflected infrared radiation via an infrared radiation detector obliquely angled relative to the longitudinal axis of the measuring chamber to receive the reflected infrared radiation; and
determining the amount of the at least one trace gas species present in the gas stream based on the infrared radiation measurement.

23. The method of claim 22, wherein the at least one trace gas species is selected from the group consisting of carbon monoxide, acetylene, and methane.

24. The method of claim 23, wherein the amount of the at least one trace gas species present in the gas stream is less than 1% by volume of the total volume of gases present in the gas stream.

25. The method of claim 22, further comprising passing the emitted infrared radiation through a first window of the measuring chamber to enter the measuring chamber.

26. The method of claim 25, further comprising passing the emitted infrared radiation through a second window of the measuring chamber to exit the measuring chamber.

27. The method of claim 22, wherein the emitting step further comprises emitting infrared radiation from at least one infrared radiation emitter, and wherein the infrared radiation emitted from each of the at least one infrared radiation emitter has a predetermined wavelength range wherein the predetermined wavelength range contains at least one peak absorbance wavelength of the at least one trace gas species.

28. The method of claim 27, further comprising configuring at least one infrared radiation measuring receiver of the infrared detector to only measure infrared radiation corresponding to one of the at least one peak absorbance wavelengths of the at least one trace gas species.

29. The method of claim 27, further comprising configuring at least one reference receiver of the infrared detector to measure infrared radiation at infrared wavelengths which do not include the at least one peak absorbance wavelengths of the at least one trace gas species.

30. The method of claim 22, wherein the step of emitting infrared radiation further comprises emitting the infrared radiation at an oblique angle relative to the longitudinal axis of the measuring chamber such that the infrared radiation is reflected off of the infrared reflective material at least three times.

31. The method of claim 22, wherein a distance of a path of travel of the emitted infrared radiation within the measuring chamber is greater than twice a height of the measuring chamber.

32. The method of claim 27, wherein the predetermined wavelength range is from 1 to 5 micrometers.

33. The method of claim 22, further comprising heating the measuring chamber.

34. The method of claim 22, wherein the infrared reflective material comprises a material with greater than 90% reflectivity in the infrared spectrum.

35. The method of claim 34, wherein the infrared reflective material is selected from the group consisting of gold, silver, aluminum, aluminum silicon oxide, and aluminum magnesium fluoride.

36. The method of claim 22, wherein the respiratory device comprises a ventilator.
